(19) United States
(12) Patent Application Publication
(10) Pub. No.: US 2013/0204102 A1
(43) Pub. Date: Aug. 8, 2013

(54) SYSTEM AND METHOD FOR NON-INVASIVE DETERMINATION OF HEMOGLOBIN CONCENTRATION IN BLOOD

(71) Applicants: Abhishek Sen, Nashik (IN); Aman Midha, Ludhiana (IN)

(72) Inventors: Abhishek Sen, Nashik (IN); Aman Midha, Ludhiana (IN)

(21) Appl. No.: 13/786,978

(22) Filed: Mar. 6, 2013

(30) Foreign Application Priority Data
Sep. 6, 2010 (IN) 2472/MUM/2010
Aug. 30, 2011 (IN) PCT/IN2011/000595

(51) Int. Cl. A61B 5/1455; A61B 5/00

(52) U.S. Cl. CPC A61B 5/14551; A61B 5/14552; A61B 5/7235
USPC 600/322

(57) ABSTRACT

A system and method for measurement of absolute value of hemoglobin concentration non-invasively is provided. The system comprises a probe device comprising a sliding top structurally configured to be manually slid forward and backward onto the finger seat which is positioned on top of the housing for placing a fingertip. The finger seat houses two cavities for housing a set of three light emitting diodes and a photodetector respectively. Multiple distinct wavelengths of light transmitted through the fingertip is detected by the photodetector. Further, electronic signals generated by the photodetector are processed to obtain alternating and direct components of light corresponding to each wavelength. A system of three equations are obtained including unknown values of two primary constituent absorbers and known consolidated values of one or more secondary constituent absorbers corresponding to each wavelength of light. The system of equations are then solved simultaneously derive absolute value of hemoglobin concentration.

### FIELD OF INVENTION

The present invention relates generally to system and method for determining hemoglobin concentration in blood. More particularly, the invention implements a process for measuring absolute value of hemoglobin concentration non-invasively.

### BACKGROUND OF THE INVENTION

Various medical conditions may require the measurement of hemoglobin concentration in blood. Hemoglobin is typically composed of constituents such as oxyhemoglobin, deoxyhemoglobin, carboxyhemoglobin, methemoglobin etc. Pulse oximetry is a commonly used non-invasive method for measuring amount of oxygen saturated hemoglobin ([0001] in tissue by transmitting infrared light through the tissue to a receiver. Saturated hemoglobin is the ratio of oxyhemoglobin to total concentration of hemoglobin in blood.

The principle of pulse oximetry is based on the fact that oxyhemoglobin and its deoxygenated form i.e. reduced hemoglobin have significantly different light absorption pattern. Typically, a conventional pulse oximeter uses two Light Emitting Diodes (LEDs), generating red and infrared lights through a part of the body. Oxygenated hemoglobin absorbs more infrared light and allows more red light to pass through, whereas deoxygenated (i.e. reduced hemoglobin) absorbs more red light and allows more infrared light to pass through. The transmitted red and infrared signals are received by a photodetector and the ratio of red signal to infrared signal is calculated. The calculated ratio is then compared to a lookup table created from previous calibrations and the ratio is then converted into an oxygen saturation level value.

In certain medical conditions such as anaemia, knowledge of absolute value of hemoglobin concentration may be desired for diagnosis and treatment. However, currently available methods for measuring absolute hemoglobin concentration value require collecting blood invasively.

In light of the above, there exists a need for a safe and accurate method for measuring hemoglobin concentration non-invasively.

### SUMMARY OF THE INVENTION

An object of the present invention is to provide a non-invasive method for determining absolute value of hemoglobin concentration in blood. In an embodiment of the present invention, the method includes directing diffused light of three distinct wavelengths through a human tissue bed in a time-multiplexed manner. Optical signal transmitted through the human tissue bed corresponding to each wavelength is then detected by a photodetector and is converted into a current signal. The current signal is then amplified to obtain a digital voltage waveform signal. By processing the digital voltage waveform signal, alternating and direct components of light transmitted through the human tissue bed are obtained. Using Beer Lambert's law and alternating and direct components of light, a system of three equations is obtained which comprise two unknown values of primary constituent absorbers of hemoglobin concentration and an unknown value of path length. Concentration values of the two primary constituent absorbers of hemoglobin concentration are derived by simultaneously solving the system of equations. However, concentration values of one or more secondary constituent absorbers are empirically derived and then combined with concentration values of the primary constituent absorbers for obtaining absolute value of hemoglobin concentration.

In various embodiments of the present invention, diffused light emitted by light emitting diodes are in near-infrared region of electromagnetic spectrum, typically within the range 600-1000 nm. The light emitting diodes and the photo detector are physically arranged in different planes.

In various embodiments of the present invention, the primary constituent absorbers of hemoglobin concentration comprise oxyhemoglobin and deoxyhemoglobin. The one or more secondary constituent absorbers comprise at least one of carboxyhemoglobin and methemoglobin.

In various embodiments of the present invention, the method steps in processing digital voltage waveform signal for obtaining distinct alternating and direct components of the signal comprise removing ambient light noise component from the digital waveform signal by digitally subtracting the ambient light noise component. Using autocorrelation, period of the waveform is then calculated and an interleaved signal is obtained by performing block interleaving for reducing errors in the digital waveform signal. Moving average filtering is applied to the interleaved signal for reducing random noise and block de-interleaving is performed for recovering the digital waveform signal in vector form. For extracting alternating and direct components of the digital waveform signal, the digital vector signal is processed using component analysis algorithm.

In an embodiment of the present invention, a probe device for facilitating direction of near-infrared light through a human tissue bed, such as a fingertip and acquiring resulting optical signal transmitted through the fingertip is claimed. The probe device comprises a housing with a flat base. Further, the probe device comprises a finger seat fastened and positioned on top of the housing for placing the fingertip and housing a first cavity and a second cavity. The first cavity is structurally arranged to house a set of three light emitting diodes and the second cavity is structurally arranged to house a photodetector. The three light emitting diodes are arranged in a non-parallel configuration and point towards a common direction while emitting light of distinct wavelengths. One or more snap holders snap fit the set of three light emitting diodes and the photodetector respectively. A sliding top is structurally configured to be manually slid forward and backward onto the finger seat. As the sliding top is slid forward over the finger seat, a pressure inducing flap applies increased pressure on the fingertip. However, as the sliding top is slid backward over the finger seat, a recoil spring structurally connected to the pressure inducing flap is configured to push the flap upward and release pressure. In an embodiment of the present invention, the three light emitting diodes are structurally arranged to be equidistant from the photodetector.

### BRIEF DESCRIPTION OF THE ACCOMPANYING DRAWINGS

The present invention is described by way of embodiments illustrated in the accompanying drawings wherein:

FIGS. 1A, 1B and 1C are illustrations depicting structure and working of probe device facilitating application of near infrared light through a fingertip and acquisition of optical signal transmitted through the fingertip, in accordance with an embodiment of the present invention.

FIG. 2 illustrates a schematic diagram for implementing acquisition and conditioning of optical signal reflected from a human tissue bed, in accordance with an embodiment of the present invention;

FIG. 3 illustrates circuit diagram of a pre-amplifier circuit;

FIG. 4 illustrates a low pass circuit configured to filter out noise from signal obtained at output of sample and hold circuit, in accordance with an embodiment of the present invention;

FIG. 5 illustrates a band pass filter circuit configured to filter out noise from signal obtained at output of sample and hold circuit, in accordance with another embodiment of the present invention;

FIG. 6 illustrates a flowchart depicting method steps for pre-processing of conditioned optical signal in order to obtain distinct values of AC and DC components of optical signal; and

FIG. 7 illustrates a flowchart depicting method steps utilizing distinct AC and DC values of optical signals for determining hemoglobin concentration levels in blood.

### DETAILED DESCRIPTION OF THE INVENTION

The disclosure is provided in order to enable a person having ordinary skill in the art to practice the invention. Exemplary embodiments herein are provided only for illustrative purposes and various modifications will be readily apparent to persons skilled in the art. The general principles defined herein may be applied to other embodiments and applications without departing from the spirit and scope of the invention. The terminology and phraseology used herein is for the purpose of describing exemplary embodiments and should not be considered limiting. Thus, the present invention is to be accorded the widest scope encompassing numerous alternatives, modifications and equivalents consistent with the principles and features disclosed herein. For purpose of clarity, details relating to technical material that is known in the technical fields related to the invention have been briefly described or omitted so as not to unnecessarily obscure the present invention.

The present invention would now be discussed in context of embodiments as illustrated in the accompanying drawings.

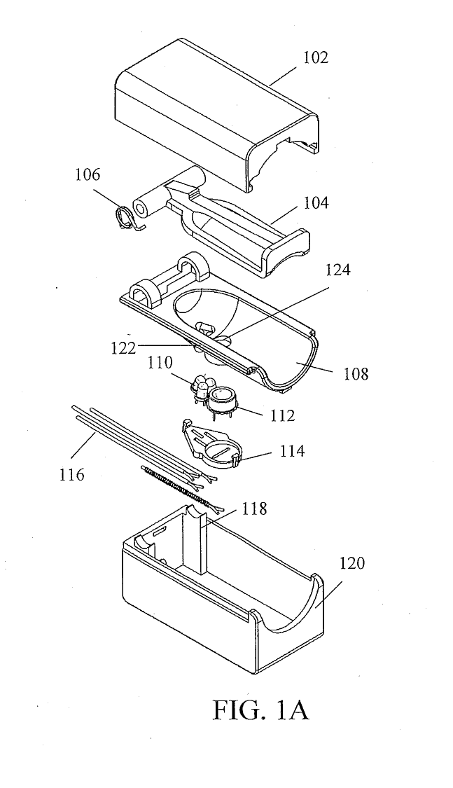

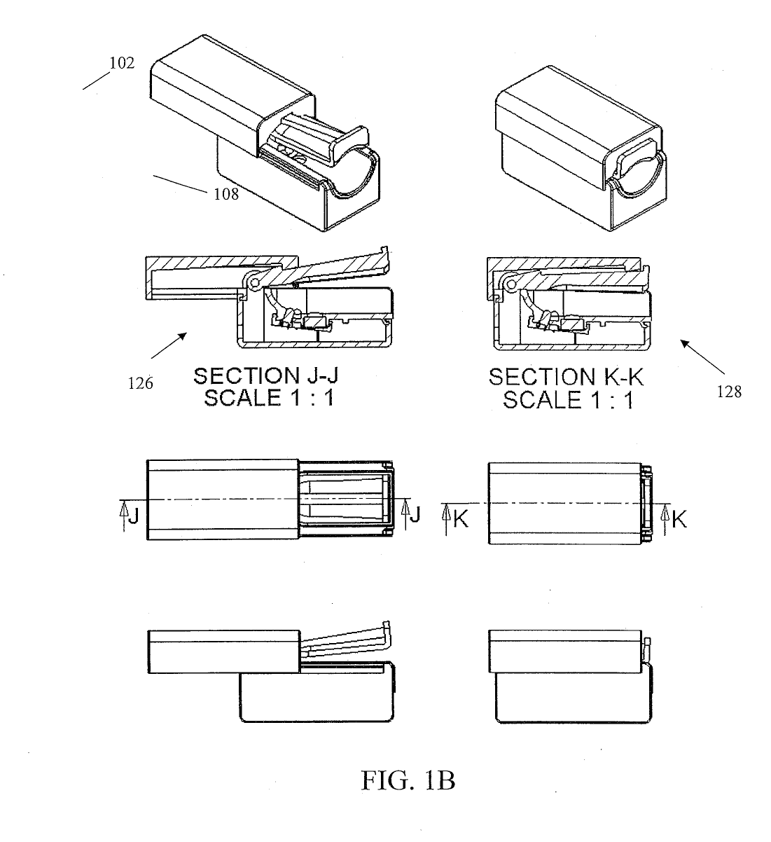

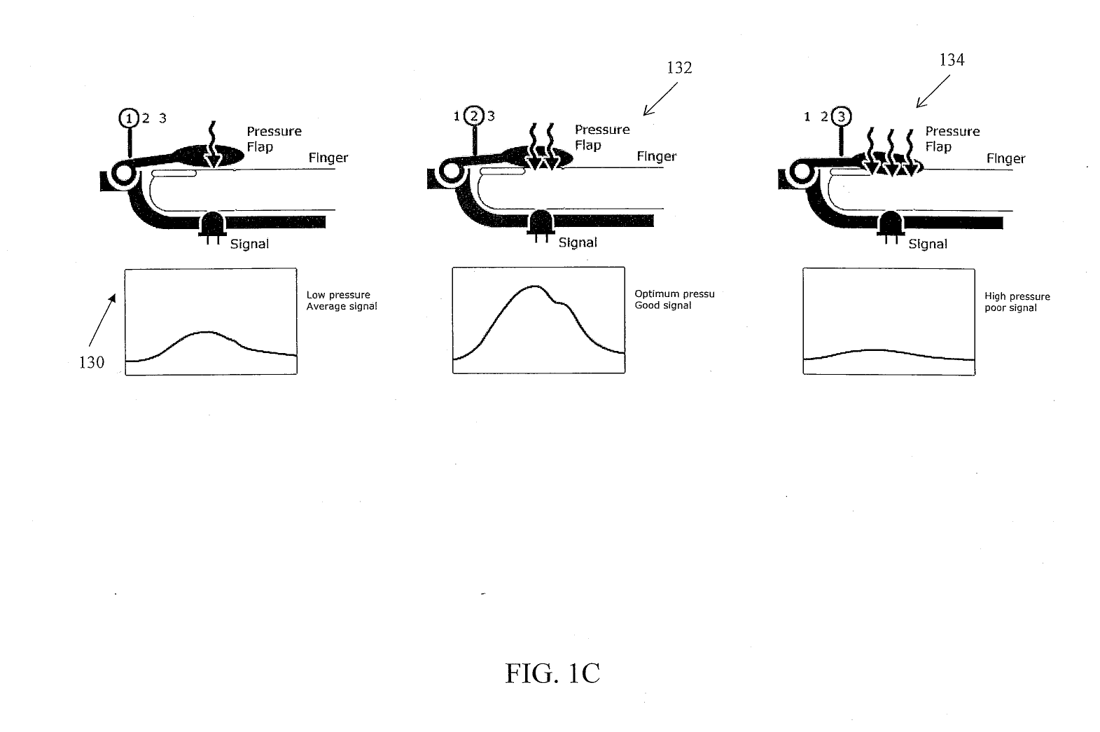

FIGS. 1A, 1B and 1C are illustrations depicting structure and working of probe device facilitating application of near infrared light through a fingertip and acquisition of optical signal transmitted through the fingertip, in accordance with an embodiment of the present invention. FIG. 1A shows exploded view of a probe device depicting individual structural components. As shown in the figure, structural components of the probe device comprises a sliding top 102, a pressure inducing flap 104 including a recoil spring 106, a finger seat 108, three light emitting diodes 110, a photodetector 112, snap emitter/photodetector holder 114, connecting wires 116, shielded wire for photodetector 118 and probe base/envelope 120. The sliding top 102 is part of probe device that moves inward and outward over the finger seat 108 and depresses the pressure inducing flap 104. The finger seat 108 houses cavities for supporting the three light emitting diodes 110 and the photodetector 112 which snap into cavities housed in the finger seat 108 using one or more snap holders 114. The finger seat is supported at the bottom by probe base/envelope 120. In an embodiment of the present invention, components of the probe device are constructed using Acrylonitrile Butadiene Styrene (ABS) plastic resin.

FIG. 1B illustrates sliding operation of the probe device. In an embodiment of the present invention, the sliding top 102 is manually slid over the finger seat 108. A human tissue bed such as a fingertip is placed on the finger seat 108. Light is directed onto the fingertip from the three light emitting diodes 110 snapped in first cavity 122. The fingertip is placed on the finger seat 108 such that the three light emitting diodes 110 are located closer towards tip of the finger. Light transmitted through the fingertip is then sensed by the photodetector 112 which is snapped in second cavity 124. The photodetector 112 coverts the sensed optical signal into a current signal to be processed later.

In order to obtain a high quality signal, there has to be sufficient pressure developed on the fingertip when light is transmitted through it. As the sliding top 102 is manually slid forward, it depresses the pressure inducing flap 104 and the flap 104 exerts pressure on the fingertip. When the sliding top 102 is moved backward, the recoil spring 106 pushes the flap 104 upward and loosens grip on the finger. Cross sectional views 126 and 128 illustrate the process of depressing the pressure inducing flap 104 as the siding top 102 is slid forward.

FIG. 1C depicts three configurations 130, 132 and 134 illustrating various stages of depression of the pressure inducing flap 104. When light is directed onto the fingertip, absorption of light by the constituents of hemoglobin is directly proportional to volume of blood passing through arteries in the fingertip. The volume of blood flow rises (peaks) with the systolic and drops to a minimum at the diastole.

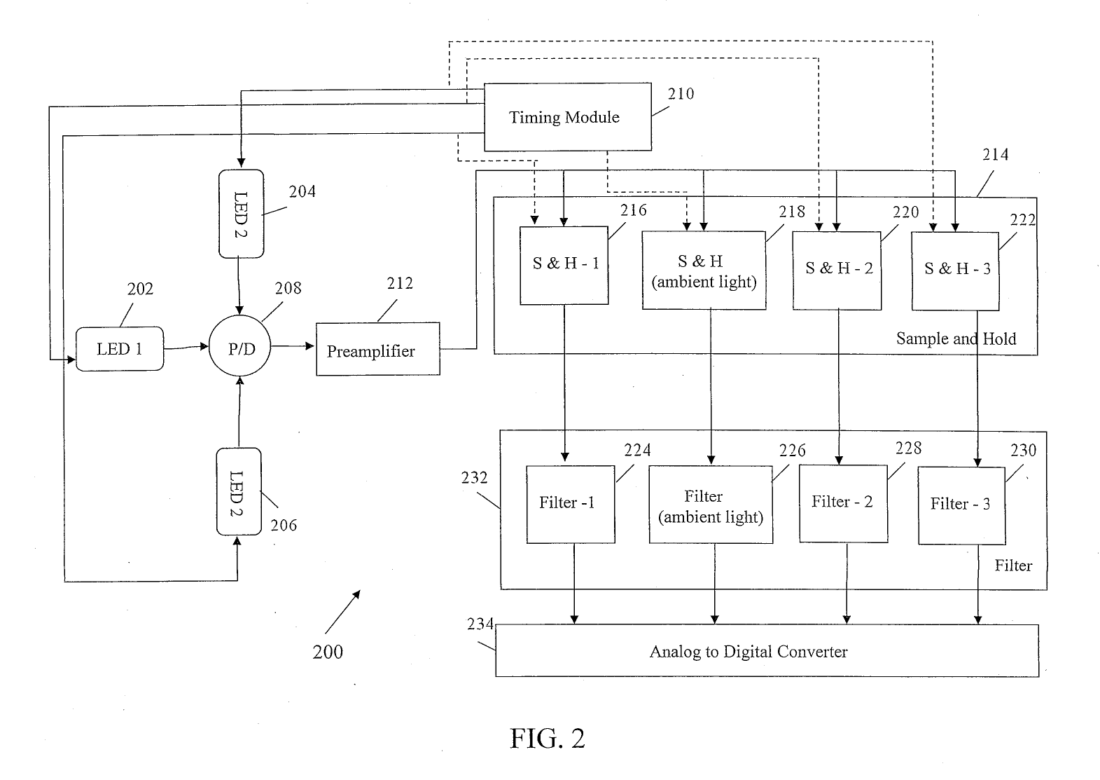

The fingertip should be depressed so that a distinct current pulse waveform corresponding to the systolic and diastolic is obtained from the photodetector 112. Configuration 130 illustrates a position where the pressure inducing flap 104 applies minimal pressure on the fingertip such that volume of blood flow through arteries in the fingertip is very less. Consequently, signal obtained through the photodetector is a low pressure signal (with indistinct maximum and minimum points). Contrastingly, configuration 134 shows application of high degree of pressure on the fingertip so that blood flow is suppressed and the current signal obtained is also of low quality. However, configuration 132 shows a moderate application of pressure so that the signal quality is improved to a point where the peak-to-peak is maximum. The signal thus obtained is optimum and is applied to a preamplifier (as described in FIG. 2) for being converted into a voltage signal for further processing.

FIG. 2 illustrates a schematic diagram 200 for implementing acquisition and conditioning of optical signal obtained by projecting light onto a human tissue bed, in accordance with an embodiment of the present invention. The present invention proposes a method for measuring hemoglobin concentration in blood non-invasively by utilizing an approach of projecting light onto a human tissue bed such as a fingertip. Light transmitted through the finger tip is then acquired and converted into an electronic signal by an electronic device. The electronic signal is then mathematically conditioned and used for measuring hemoglobin concentration. A preferred embodiment of the present invention uses the principle of transmittance spectrophotometry for measurement of hemoglobin concentration. As per the aforementioned principle, three Light Emitting Diodes (LEDs) are used for projecting light onto a human fingertip. Light transmitted through the fingertip is detected by a photodetector and analog signals are generated which are used for calculating absolute value of hemoglobin concentration in blood. An LED is a semiconductor device that uses the principle of electroluminescence in order to emit light to be directed onto the finger. LED is a diffuse light source that releases several photon packets over a large surface area. Since human tissue comprises multiple light scattering elements, the diffused light activates scattering and back-scattering of photons within the human tissue. Further, the LEDs are positioned such that they are equidistant from the photodetector and have similar optical and physical characteristics such as size and half viewing angle. Due to the scattering and back-scattering of photons, light penetration characteristic through human tissue is random. As a result, mathematically, light penetration through human tissue can be considered to have a normal or Gaussian distribution. Due to this effect and the orientation of LEDs, distance travelled by light through various constituents of blood can be assumed to be constant. In various embodiments of the present invention, this property is utilized for calculating absolute value of hemoglobin concentration in blood.

The schematic diagram 200 illustrates three Light Emitting Diodes (LEDs) 202, 204 and 206 positioned to direct light through a finger in the probe device. The LEDs 202, 204 and 206 are part of a probe device constructed to direct light onto a human tissue bed, such as a fingertip. In an embodiment of the present invention, electric current is passed through each LED such that each LED shines light on the finger at a distinct wavelength. In various embodiments of the present invention, toes or ear lobes may also be used instead of a fingertip as a human tissue bed. The LEDs 202, 204 and 206 are configured to emit light in near-infrared region of electromagnetic spectrum. Part of the emitted light is absorbed by blood constituents and part of it is transmitted through human tissue and is detected by a photodetector 208. Primarily, the projected light is absorbed by various forms of the oxygen-containing metalloprotein hemoglobin. Various forms of hemoglobin that absorb the projected light include oxyhemoglobin, deoxyhemoglobin, carboxyhemoglobin and methemoglobin. For each wavelength of light impinged on the fingertip, the photodetector 208 converts the transmitted optical signal into a current signal which is then mathematically processed in order to determine an absorbance value. In various embodiments of the present invention, the photodetector may be one of a photodiode, a phototransistor or a photoresistor. Absorbance is defined as ratio of measure of radiant light absorbed by blood constituents to measure of incident light. Using absorbance values at each wavelength and the principle of Beer-Lambert's law, absolute value of concentration of hemoglobin in blood can be determined.

Utilizing an optical signal transmitted through human tissue for non-invasively determining bodily constituents is known as transmittance spectrophotometry. One of the advantages of using transmittance spectrophotometry is that optical signal obtained due to transmittance is a high amplitude signal. The high amplitude signal is easier to process mathematically for obtaining parameters used in the determination of hemoglobin concentration in blood. For obtaining a transmitted optical signal, the LEDs 202, 204 and 206, and the photodetector 208 are located in different planes. As stated earlier, an important feature of the present invention is the use of near-infrared region of electromagnetic spectrum. Near-infrared region is the region of light with wavelengths in the range 600-3000 nm. In an embodiment of the present invention, wavelengths in the range 600-1000 nm are used. This is because light penetration decreases as wavelength increases, e.g. 1300 nm will penetrate less than 660 nm. However, in the window of 600-1000 nm, penetration is relatively constant. After penetrating human tissue, components of the projected light are absorbed, scattered, transmitted or reflected by arteries. Primary component in an artery that contributes to absorbing the projected light is blood flowing through the artery. Apart from fraction of incident light being absorbed, portions of the incident light get scattered due to the presence of light scattering components such as melanin in human skin. Light which is neither absorbed, scattered or back scattered due to reflection, is transmitted through the finger and is detected by the photodetector 208. The photodetector 208 converts the transmitted optical signal into current signal. The current signal is then mathematically processed. In an embodiment of the present invention, reflectance spectrophotometry may be used instead of transmittance spectrophotometry for the determination of hemoglobin concentration. Reflectance spectrophotometry may be used, in an embodiment, since it is more robust to motion artifacts. The LEDs 202, 204 and 206, and the photodetector 208 may be physically arranged in the same plane and light reflected from the finger is used to obtain an electronic signal which is mathematically processed to determine hemoglobin concentration in blood.

In various embodiments of the present invention, current signal corresponding to a wavelength obtained from the photodetector 208 is mathematically processed so as to obtain Alternating-Current (AC) and Direct Current (DC) components of light of each wavelength directed by the LEDs 202, 204 and 206. In an embodiment of the present invention, light emission by the LEDs 202, 204 and 206 are time multiplexed. Time multiplexing is done so as to obtain independent optical signals which are processed to determine absorbance values related to various forms of hemoglobin at three wavelengths. In an embodiment of the present invention, time multiplexing of light emission is realized by a Timing module 210. In an embodiment of the present invention, the Timing module 210 comprises a standard 555 timer circuit. In another embodiment of the present invention, the Timing module 210 employs a microcontroller. Using the AC and DC components of light at each wavelength, absorbance values at each wavelength are determined by the formula:

Absorbance=log 10([0001] (1)

Ambient light interferes with light emitted by the LED's 202, 204 and 206. As a result, light captured by the photodetector 208 contains not only a component of unabsorbed light as emitted by the LED's but also a component of unabsorbed ambient light. Thus, in an embodiment of the present invention, in addition to processing optical signals corresponding to three wavelengths, an optical signal corresponding to ambient light is also processed by the system of the present invention.

Referring now to the figure, current signal obtained from the photodetector 208 corresponding to each wavelength and the ambient light is converted into a voltage signal by a preamplifier 212. In an embodiment of the present invention, preamplifier 212 is a standard current-to-voltage preamplifier with low noise and high input impedance and configured to amplify and convert low amplitude current signals into voltage signals. Since current signal obtained from the photodetector 208 typically has low amplitude, the preamplifier 212 with low noise is able to effectively amplify the signal. Output from the photodetector 208 is provided to a sample and hold module 214. As shown in the figure, voltage signal corresponding to each wavelength and the ambient light is applied to independent sample and hold circuits 216, 218, 220 and 222. A sample and hold circuit is an electronic circuit configured to hold analog voltage signal at each wavelength steady for further processing of signal to take place. Steady voltage signals at the output of sample and hold circuits 216, 218, 220 and 222 are applied to individual filter circuits 224, 226, 228 and 230 in a filter module 232. The filter circuits 224, 226, 228 and 230 operate to suppress unwanted noise, such as static from other devices, 50/60 Hz line interference that may accompany voltage signals at the output of sample and hold circuits 216, 218, 220 and 222. Output analog signals from the filter module 232 are provided to an Analog to Digital (A/D) converter 234. The A/D converter 232 converts analog signals corresponding to the three wavelengths and the ambient light into digital signals to be processed later.

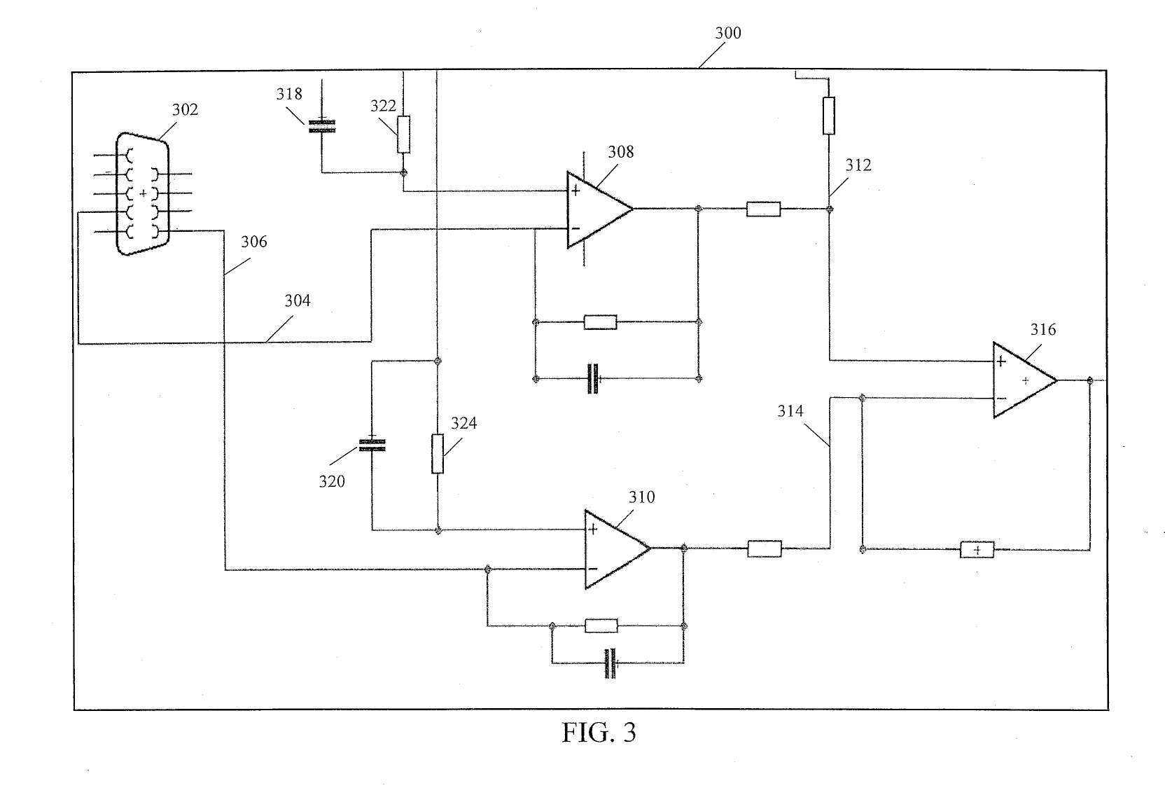

FIG. 3 illustrates circuit diagram of a pre-amplifier circuit 300, in accordance with an embodiment of the present invention. The pre-amplifier circuit takes current signals corresponding to light directed on human tissuebed as inputs and converts the signals into voltage signals. As shown in the figure, a standard converter 302 receives current signal as input from a photodetector and provides the signal to transimpedence amplifiers in order to generate an amplified voltage signal. Outputs 304 and 306 obtained from anode and cathode of a photodetector respectively are applied to operational amplifiers 308 and 310 operating in an inverting configuration for generating corresponding voltage signals 312 and 314. The voltage signals 312 and 314 are then presented to a differential amplifier 316 for obtaining an amplified output signal. In an embodiment of the present invention, capacitors 318 and 320 are connected across input resistances 322 and 324 to suppress electrical noise.

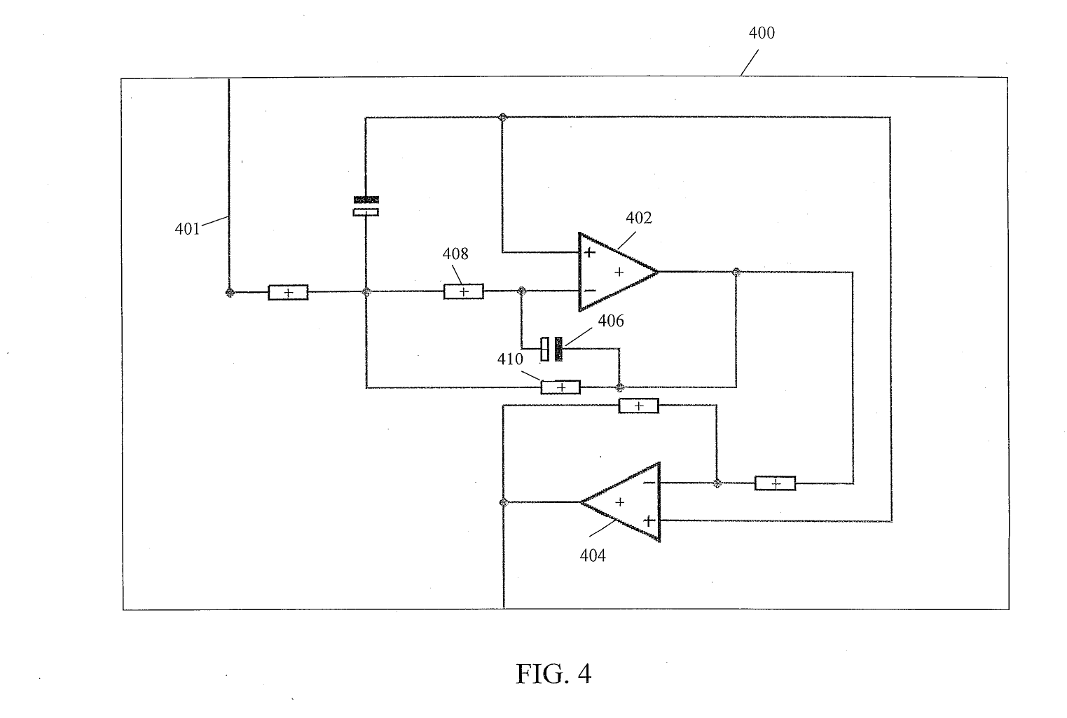

FIG. 4 illustrates a low pass circuit 400 configured to filter out noise from signal obtained at output of sample and hold circuit, in accordance with an embodiment of the present invention. The circuit comprises two operational amplifiers 402 and 404 operating to filter out high frequency noise. Input from a sample and hold circuit is applied to an input node 401. In an exemplary embodiment of the present invention, values of capacitor 406 and the resistors 408 and 410 are chosen such that the filter operates at a corner frequency of approximately 5 Hz. Thus the low pass filter 400 functions to filters out high frequency noises such as static noise from other devices.

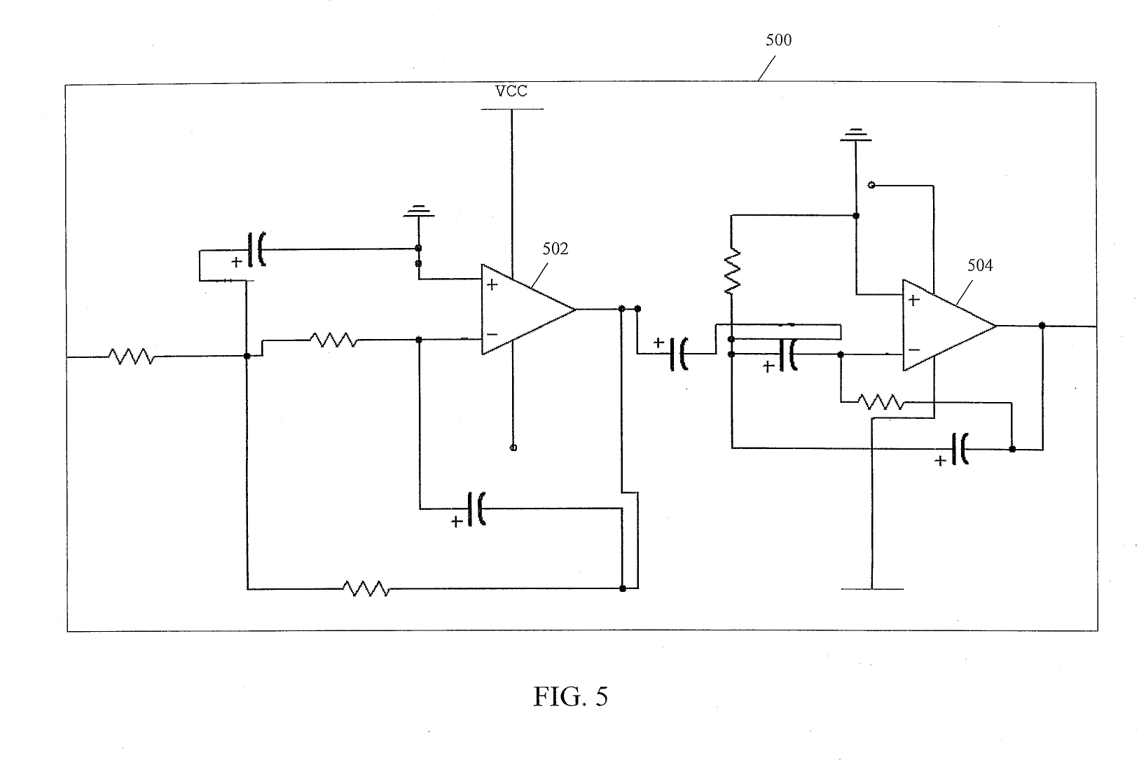

FIG. 5 illustrates a band pass filter circuit 500 configured to filter out noise from signal obtained at output of sample and hold circuit, in accordance with another embodiment of the present invention. Instead of using a low pass filter circuit (illustrated in FIG. 4), the band pass filter circuit 500 may be used for selective filtering. In an exemplary embodiment of the present invention, the operational amplifiers 502 and 504 operate to only pass through signals in the frequency range 0.5-5 Hz.

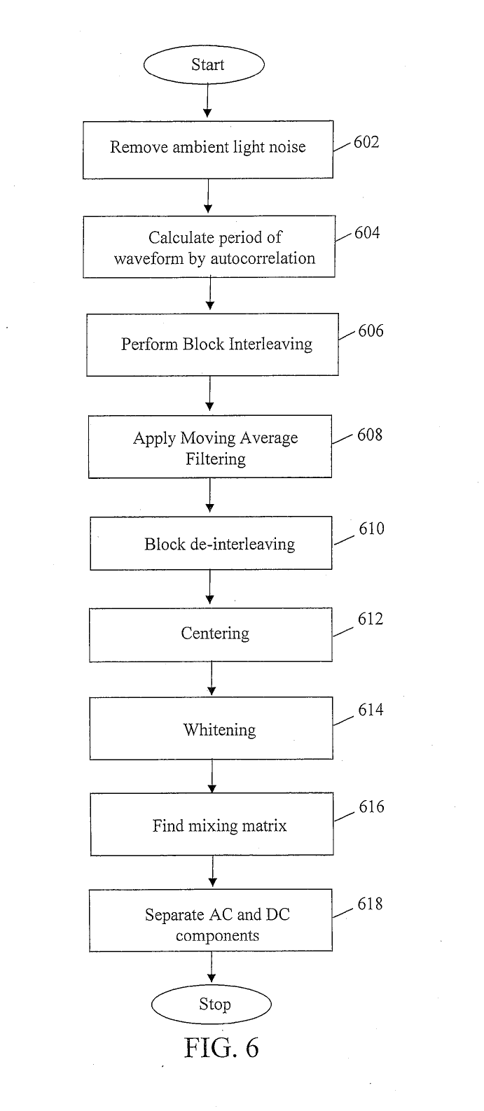

FIG. 6 illustrates a flowchart depicting method steps for pre-processing of conditioned optical signal in order to obtain distinct values of AC and DC components of optical signal. As described with reference to FIG. 2, signal corresponding to each wavelength of light transmitted through a human tissuebed and the ambient light signal are converted to digital signals using an analog to digital converter. The digital signal thus obtained is a digital waveform comprising both AC and DC components of an optical signal which are then processed further so as to isolate the AC and DC components. Processing of each digital signal begins at step 602. Ambient light is first subtracted from each digital signal in order to nullify effect of noise due to ambient light. Since the digital waveform obtained comprises both data associated with light transmitted through the human tissue bed as well as external noise, autocorrelation is performed at step 604 to obtain period of waveform that includes relevant data. Autocorrelation is a mathematical process for analyzing time domain signals, wherein degree of similarity between a time domain signal and a lagged version of the signal over successive time intervals is ascertained. In an embodiment of the present invention, using degree of similarity between a time domain signal and lagged version of the signal over successive time intervals, period of the waveform can be determined. Following the determination of period of waveform, the waveform is further processed in order to isolate the AC and DC components. In another embodiment of the present invention, instead of determining period of waveform, frequency of the waveform can be determined using cepstrum analysis and the resulting signal is then processed for isolating AC and DC components. Cepstral analysis involves taking fourier transform of the time domain waveform, taking logarithm of the fourier transform and then taking fourier transform of the resulting signal. The process of cepstral analysis can be explained as follows:

Let W be a digital waveform obtained corresponding to each wavelength of light.

The process steps in performing cepstral analysis are as follows:

A discrete fourier transform of the waveform is first obtained as illustrated by the equation:

X=DFT(waveform) (2)

A cepstral signal is then obtained using the following equation:

CP=DFT(log {abs(X)}) (3)

Following the determination of relevant period of the waveform or a cepstral signal (as the case may be), at step 606, block interleaving is performed in order to reduce errors. Block interleaving is a process of rearranging data by reordering individual characters in a structured manner so that errors are reduced. In an embodiment of the present invention, block interleaving is done by arranging coordinates of the digital waveform into an N×M matrix.

With reference to the present invention, following the process of block interleaving, at step 608, moving average filter is applied to the output of block interleaver for reducing random noise. A standard low pass filter may be used as a moving average filter. At step 610, block de-interleaving is performed to recover the digital waveform in vector form. In an embodiment of the present invention, after block de-interleaving, the digital vector is processed using Independent Component Analysis (ICA) algorithm. An ICA algorithm is a statistical algorithm for decomposing a complex dataset into independent sub-parts. A primary step in executing the ICA algorithm is to first preprocess the digital vector. Preprocessing includes centering the digital vector at step 612. Centering is performed to simply the digital vector into a zero-mean vector. After centering, whitening is performed at step 614 by transforming the digital vector linearly so as to obtain a whitened vector (having uncorrelated components and variances equal to 1). In an embodiment of the present invention, at step 616, a fast ICA algorithm is may be used for determining a mixing matrix. Thus independent components are calculated that are added to mean vector of each of the independent components. After calculating the independent components, AC and DC components of the digital waveform are obtained at step 618. The AC and DC components of the digital waveform correspond to the AC and DC components of the optical signal transmitted through the human tissue bed. The above process steps are repeated for each wavelength of light to independently obtain AC and DC components corresponding to each wavelength. In another embodiment of the present invention, after obtaining a whitened vector at step 614, instead of using a fast ICA algorithm, a Principal Component Analysis (PCA) algorithm is used to obtain AC and DC components of digital waveform. A PCA algorithm implements search and extraction of principal components of a mixed waveform. For implementing the PCA algorithm, eigen values and eigen vectors corresponding to the digital vector are first calculated and then eigen vectors with higher eigen values are chosen as principal components for obtaining AC and DC components of the digital waveform.

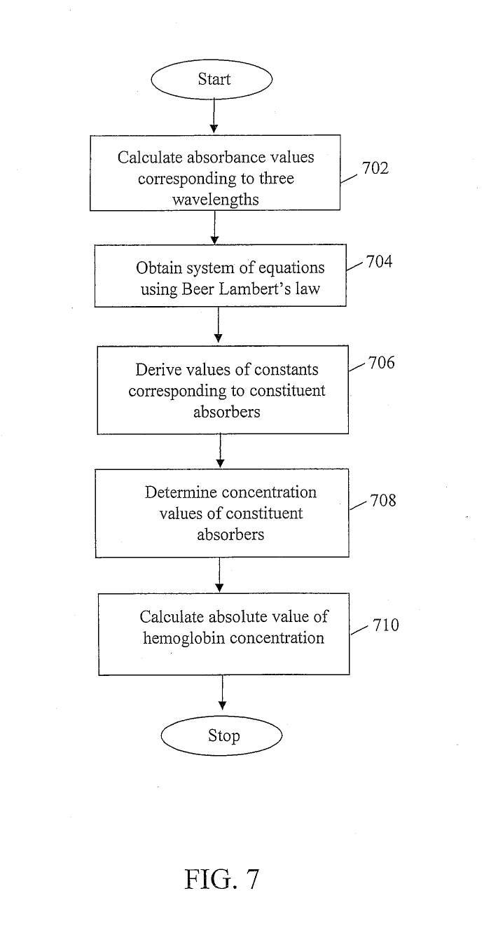

FIG. 7 illustrates utilizing distinct AC and DC values of optical signals for determining hemoglobin concentration levels in blood. In an embodiment of the present invention, at step 702 three absorbance values corresponding to three distinct wavelengths of light are calculated using the AC and DC values of optical signals using mathematical equation (1). Thus three distinct absorbance values are obtained corresponding to the three wavelengths of light. These absorbance values are then applied in a standard Beer Lambert's law equation for determining concentration of hemoglobin. According to Beer Lambert's law, absorbance is proportional to thickness of a sample as well as concentration of absorbing species in the sample. Since hemoglobin comprises multiple constituents such as oxyhemoglobin, deoxyhemoglobin, carboxyhemoglobin, methemoglobin etc., each of these constituents absorb incident light depending upon their individual concentrations.

The following equation illustrates Absorbance as a function of concentration of an absorber:

Absorbance=Distance travelled by light×Extinction Coefficient×Concentration (4)

where distance travelled by light is the length of path through which light travels to reach an absorber, concentration is the concentration of absorber and extinction coefficient is the molar absorptivity of the absorber i.e. how strongly an absorber absorbs light at a particular wavelength. As explained earlier in the description of FIG. 2, since a diffused light source is used for transmitting light through a fingertip, light penetration can be considered to be normal and thereby distance travelled by light through various constituents of blood can be assumed to be constant for different wavelengths of light. There are a number of active constituents in blood that absorb near-infrared light i.e. oxyhemoglobin, reduced hemoglobin, carboxyhemoglobin, methemoglobin, lipids etc. Now, assuming n number of constituent absorbers in blood, for a particular wavelength of light, absorbance can be defined by the following equation:

Absorbance=d(E1*C1+E2*C2+E3*C3+E4*C4+E5*C5 . . . +En* Cn) (5)

where d is path length through which incident light travels, E1 and C1 are extinction coefficient and concentration of first constituent absorber, E2 and C2 are extinction coefficient and concentration of second constituent absorber, E3 and C3 are extinction coefficient and concentration of third constituent absorber and so on. However, the majority of total hemoglobin in blood is comprised primarily of two constituent absorbers i.e. oxyhemoglobin and deoxyhemoglobin (reduced hemoglobin). Hence, in an embodiment of the present invention, absorbance corresponding to a particular wavelength of light can be illustrated by the following equation:

Absorbance=d(E11*C1+E12*C2+K1) (6)

where K1 is a constant representing collective value secondary constituent absorbers C3 to Cn. Value of K1 can be derived using either clinical calibration or parameter estimation matrix.

In various embodiments of the present invention, three equations corresponding to three wavelengths of light are obtained at step 704, as follows:

Abs1_{=d}(E11*C1+E12*C2+K1) (7)

Abs2=d(E21*C1+E22*C2+K2) (8)

Abs3=d(E31*C1+E32*C2+K3) (9)

In the above three equations, K1 K2 and K3 are constants corresponding to three wavelengths of light. In an application based on a system of linear equations with constant scaling factors, such as the above, it is not possible to get a unique, non-zero solutions for variables C 1 and C2. In this case constants representing consolidated values of secondary variables if included in the equations (7), (8) and (9) can solve the system for unique, non-zero results. Hence, values for K1, 2 and K3 are empirically derived at step 706 either from clinical calibration or from parameter estimation matrix. Further, the extinction coefficients E11, E12, E21, E22, E31 and E32 corresponding to the constituent absorbers C1 and C2 are already known and the absorbance values Abs1, Abs2 and Abs3 are calculated using equation (1). Thus, in the above three equations, the only unknown parameters are path length d and values of constituent absorbers C1 and C2. Thus, at step 708, by simultaneously solving the equations (7), (8) and (9) using available information, concentration values of constituent absorbers C1 and C2 are obtained. Adding concentration values of constituent absorbers C1 and C2, absolute value of hemoglobin concentration is obtained at step 710.

While the exemplary embodiments of the present invention are described and illustrated herein, it will be appreciated that they are merely illustrative. It will be understood by those skilled in the art that various modifications in form and detail may be made therein without departing from or offending the spirit and scope of the invention as defined by the appended claims.

1. A method for non-invasively determining absolute value of hemoglobin concentration in blood, the method comprising:
directing diffused light of three or more distinct wavelengths through a human tissue bed in a time-multiplexed manner;
detecting optical signal corresponding to each wavelength by a photodetector and converting transmitted portion of optical signal into current signal;
amplifying current signal corresponding to each wavelength of light to obtain a digital voltage waveform signal;
processing the digital voltage waveform signal to obtain alternating and direct components of light corresponding to each wavelength;
obtaining a system of three equations using alternating and direct components of light corresponding to each wavelength and Beer Lambert's law, wherein the equations comprise two unknown values of primary constituent absorbers of hemoglobin concentration and an unknown value of path length;
deriving concentration values of the two primary constituent absorbers of hemoglobin concentration; and
combining concentration values of the two primary constituents and empirically derived values of one or more secondary constituent absorbers of hemoglobin concentration in order to determine absolute value of hemoglobin concentration the one or more secondary constituent absorbers comprise at least one of carboxyhemoglobin and methemoglobin,
wherein the system of equations comprise known constants representing consolidated values of the one or more secondary constituent absorbers corresponding to each wavelength of light, the known constants are derived empirically by clinical calibration and/or parameter estimation matrix.

2. The method of claim 1, wherein diffused light of three distinct wavelengths is obtained by using three light emitting diodes emitting light in near-infrared region of electromagnetic spectrum.

3. The method of claim 2, wherein the light emitting diodes and the photodetector are physically arranged in different planes.

4. The method of claim 2, wherein the light emitting diodes and the photodetector are physically arranged in the same plane.

5. The method of claim 2, wherein light emitted by the light emitting diodes is in the range 600-1000 nm.

6. The method of claim 1, wherein the photodetector is one of a photodiode, a phototransistor or a photoresistor.

7. The method of claim 1, wherein current signal amplified to obtain digital waveform signal is obtained by converting optical signal reflected through the human tissue bed.

8. The method of claim 1, wherein the primary constituent absorbers of hemoglobin concentration comprise oxyhemoglobin and deoxyhemoglobin.

9. The method of claim 1, wherein processing the digital voltage waveform signal for obtaining distinct alternating and direct components of the signal comprises:
removing ambient light noise component from the digital waveform signal by digitally subtracting the ambient light noise component;
calculating period of waveform by autocorrelation;
obtaining an interleaved signal by performing block interleaving for reducing errors in the digital waveform signal;
applying moving average filtering to the interleaved signal for reducing random noise;
performing block de-interleaving for recovering the digital waveform signal in vector form;
processing the digital vector signal using component analysis algorithm in order to extract alternating and direct components of the digital waveform signal, wherein the alternating and direct components correspond to alternating and direct components of the optical signal; and
extracting alternating and direct components of the digital waveform signal.

10. The method of claim 9, wherein block interleaving is performed by arranging coordinates of the digital waveform signal in matrix form.

11. The method of claim 9, wherein moving average filtering is performed using a low pass filter.

12. The method of claim 9, wherein the digital vector signal is processed using fast independent component analysis algorithm.

13. The method of claim 9, wherein the digital vector signal is processed using principal component analysis algorithm.

14. The method of claim 1, wherein processing the digital voltage waveform signal for obtaining distinct alternating and direct components of the signal comprises:
removing ambient light noise component from the digital waveform signal by digitally subtracting the ambient light noise component;
determining frequency of the waveform using cepstrum analysis;
obtaining an interleaved signal by performing block interleaving for reducing errors in the digital waveform signal;
applying moving average filtering to the interleaved signal for reducing random noise;
performing block de-interleaving for recovering the digital waveform signal in vector form;
processing the digital vector signal using component analysis algorithm in order to extract alternating and direct components of the digital waveform signal wherein the alternating and direct components correspond to alternating and direct components of the optical signal; and
extracting alternating and direct components of the digital waveform signal.

15. A probe device for non-invasively determining absolute value of hemoglobin concentration in blood, the probe device comprising:
a housing with a flat base;
a finger seat fastened and positioned on top of the housing for placing the fingertip and housing a first cavity and a second cavity, wherein the first cavity is structurally arranged to house a set of three light emitting diodes and the second cavity is structurally arranged to house a photodetector, further wherein the three light emitting diodes are arranged in a non-parallel configuration and point towards a common direction, further wherein the three light emitting diodes emit light of distinct wavelengths;
one or more snap holders for snap fitting the set of three light emitting diodes and the photodetector respectively, wherein the three light emitting diodes have similar optical and physical characteristics,
a sliding top structurally configured to be manually slid forward and backward onto the finger seat;
a pressure inducing flap fastened on one side of the finger seat and configured to apply increased pressure on the fingertip as the sliding top is slid forward over the finger seat, wherein based on the application of pressure on the fingertip, current signal is generated by the photodetector proportional to light transmitted through the fingertip; and
a recoil spring structurally connected to the pressure inducing flap and configured to push the flap upward and release pressure as the sliding top is slid backward over the finger seat.

16. The probe device of claim 15, wherein the three light emitting diodes are structurally arranged to be equidistant from the photodetector.
